(19) United States
(12) Patent Application Publication
(10) Pub. No.: US 2017/0027982 A1
(43) Pub. Date: Feb. 2, 2017

(54) ANTICOAGULANT AND DECOAGULANT METHODS, COMPOSITIONS AND DEVICES

(71) Applicant: Ariel-University Research and Development Company Ltd., Ariel (IL)

(72) Inventor: Danny BARANES, Beer-Sheva (IL)

(21) Appl. No.: 15/303,029

(22) PCT Filed: Apr. 29, 2015
(86) PCT No.: PCT/IL2015/050447

§ 371 Date: Oct. 10, 2016

Related U.S. Application Data
(60) Provisional application No. 61/985,484, filed on Apr. 29, 2014.

(51) Int. Cl. A61K 33/10; A61K 9/14

(52) U.S. Cl. CPC A61K 33/10; A61K 9/14

(57) ABSTRACT

Disclosed are anticoagulant and decoagulant methods, compositions and devices comprising calcium carbonate, particularly, comprising aragonite.

### FIELD AND BACKGROUND OF THE INVENTION

The invention relates to the fields of medicine and medical research, and more particularly to methods, compositions and devices for reducing the coagulation of blood, for example anticoagulants for reducing the rate of clotting of blood and decoagulants suitable for assisting in a process of dissolution of clotted blood.

In the animal kingdom, blood transports materials such as nutrients, oxygen, metabolic waste products, cells and signaling chemicals between portions of an animal. When the animal is wounded a process of hemostasis occurs whereby the blood clots as a first step in wound healing to reduce the amount of blood lost from the animal. During hemostasis the blood coagulates to form blood clots. Clotting involves two cascades: The intrinsic cascade (which has less in vivo significance in normal physiological circumstances than the extrinsic cascade) is initiated when contact is made between blood and exposed negatively charged surfaces. The extrinsic pathway is initiated upon vascular injury which leads to exposure of tissue factor, TF (also identified as factor III), a subendothelial cell-surface glycoprotein that binds phospholipid. The two pathways converge at the activation of factor X to Xa. Factor Xa has a role in the further activation of factor VII to VIIa. Active factor Xa hydrolyzes and activates prothrombin to thrombin. Thrombin can then activate factors XI, VIII and V furthering the cascade. Ultimately the role of thrombin is to convert fibrinogen to fibrin and to activate factor XIII to XIIIa. Factor XIIIa (also termed transglutaminase) cross-links fibrin polymers solidifying the clot.

Clots, once formed, usually eventually undergo dissolution. Release of tissue plasminogen activator (tPA) from vascular endothelial cells leads to the onset of the dissolution of fibrin clots. Low levels of circulating tPA are kept inactive by interaction with various inhibitors, where plasminogen activator inhibitor-1 and -2 (PAI-1 and PAI-2) are the most significant. tPA is also removed from the circulation by hepatic cell uptake. The clot dissolving enzyme, plasminogen, binds to the fibrin clot as the inactive zymogen. Once tPA interacts with plasminogen it hydrolyzes the protein releasing catalytically active plasmin. Plasmin then can hydrolyze the cross-linked fibrin polymers of the clot resulting in its dissolution (breakdown), or “decoagulation”. Excess plasmin is controlled from over activity via interaction, in the plasma, with plasmin inhibitors such as alpha₂-antiplasmin.

Blood coagulation is not always desirable. In thrombosis, a blood clot forms in the body and can be released as a free thromboembolus into the circulation to eventually block an artery leading to the brain or heart (embolism), with grievous consequences.

However, while prevention of clot formation, or anti-coagulant activity, and dissolution of already formed clots (thrombolytic activity) is desirable in a variety of indications, most thrombolytic or anti-coagulant therapeutic compositions comprising plasminogen activator, streptokinase or urokinase, when administered systemically are accompanied by increased risk of bleeding complications, such as GI and intracranial hemorrhage.

One approach to avoiding the complications of systemic anticoagulant therapy has been the administration of plasmin or plasmin compositions directly into, or proximally to a thrombus (see, for example, US Patent Publication 20030175264 to Jesmok et al). Purely mechanical approaches for disruption and removal of clots have also been proposed (see, for example, US Patent Publication 20060253145 to Lucas and U.S. Pat. No. 5,928,218), as well as the use of ultrasonic energy (sonothrombolysis) with or without additional thrombolytic compositions (see, for example, US Patent Publication No. 20130331738 to Borrelli and 20110288449 to Schenkengel).

### SUMMARY OF THE INVENTION

The present invention relates to the fields of medicine and medical research, and more particularly to methods, compositions and devices for influencing the coagulation of blood, especially mammalian blood, for example anticoagulants for reducing the rate of clotting of blood and decoagulants suitable for assisting in a process of dissolution of clotted blood.

According to an aspect of some embodiments of the present invention there is provided a method for influencing coagulation of blood comprising contacting an effective amount of calcium carbonate with blood, thereby influencing coagulation of the blood.

According to an aspect of some embodiments of the present invention there is provided a use of particulate calcium carbonate in the preparation of a medicament for influencing the coagulation of blood.

According to some embodiments of the invention the calcium carbonate comprises crystalline calcium carbonate.

According to some embodiments of the invention the calcium carbonate comprises aragonite.

According to some embodiments of the invention the calcium carbonate comprises acellular coral exoskeleton.

According to some embodiments of the invention the coral exoskeleton is provided in dimensions which maintain a porous coral exoskeleton structure.

According to some embodiments of the invention the blood is mammalian blood.

According to some embodiments of the invention the blood is human blood.

According to some embodiments of the invention at least some of the blood is unclotted, and the contacting the calcium carbonate inhibits coagulation of the unclotted blood.

According to some embodiments of the invention at least some of the blood is clotted blood, and contacting the calcium carbonate at least partially dissolves the clotted blood.

According to some embodiments of the invention the contacting of calcium carbonate with the blood is effected in vivo.

According to some embodiments of the invention the contacting is effected in an ischemic tissue or a tissue region which is at risk of ischemia.

According to some embodiments of the invention the contacting of calcium carbonate with blood is effected by applying calcium carbonate to a wound.

According to some embodiments of the invention the contacting is effected by a method selected from the group consisting of direct application and injection.

According to some embodiments of the invention the tissue is selected from the group consisting of brain tissue, retina, skin tissue, hepatic tissue, pancreatic tissue, bone, cartilage, connective tissue, blood tissue, muscle tissue, cardiac tissue, gastrointestinal tissue, vascular tissue, renal tissue, pulmonary tissue, gonadal tissue, hematopoietic tissue.

According to some embodiments of the invention the calcium carbonate is comprised in a device.

According to some embodiments of the invention the device comprising the calcium carbonate is selected from the group consisting of a catheter, a dialysis catheter, a balloon catheter and an embolic protection device.

According to some embodiments of the invention the contacting comprises implanting the device in a body of a living organism.

According to some embodiments of the invention the device is selected from the group consisting of a stent, a heart valve, a blood vessel bypass, a blood vessel graft, a filter, an artificial heart, a shunt and an embolic protection device.

According to some embodiments of the invention the contacting of the calcium carbonate with the blood is effected ex vivo.

According to some embodiments of the invention, the method comprises ex vivo contacting the blood with a device comprising the calcium carbonate.

According to some embodiments of the invention the device is selected from the group consisting of an artificial heart, a renal dialysis device, a pheresis device and transfusion filter.

According to some embodiments of the invention the contacting of the calcium carbonate with the blood is effected in vitro.

According to some embodiments of the invention the method comprises in vitro contacting the blood with a device comprising the calcium carbonate.

According to some embodiments of the invention the device is selected from the group consisting of blood collection or storage device, a conduit, a flask, a bottle, a dish, a petri dish, a plate, a multiwell plate, a test tube and a blood transfusion bag.

According to an aspect of some embodiments of the present invention there is provided a device for contacting blood, comprising isolated, disinfected aragonite.

According to some embodiments of the invention the aragonite comprises coral exoskeleton.

According to some embodiments of the invention the device is configured for placement inside the body of a living animal and contact with blood or in vivo.

According to some embodiments of the invention the device is selected from the group consisting of a catheter, a dialysis catheter, a balloon catheter, an embolic protection device, a stent, a heart valve, a blood vessel bypass, a blood vessel graft, a filter, an artificial heart, a shunt and an embolic protection device.

According to some embodiments of the invention the device is configured for contact with blood ex vivo.

According to some embodiments of the invention the device is selected from the group consisting of an artificial heart, renal dialysis device and transfusion filter, a blood collection or storage device, a conduit, a flask, a bottle, a dish, a petri dish, a plate, a multiwell plate, a test tube and a blood transfusion bag.

According to an aspect of some embodiments of the present invention there is provided a pharmaceutical composition for inhibiting the coagulation of blood and/or dissolving clotted blood, comprising particulate calcium carbonate and a pharmaceutically acceptable carrier.

According to some embodiments of the invention the particulate calcium carbonate comprises aragonite.

According to some embodiments of the invention the particulate calcium carbonate comprises coral exoskeleton.

According to some embodiments of the invention the particulate calcium carbonate is disinfected, medical grade aragonite.

According to some embodiments of the invention the pharmaceutical composition further comprises an additional active ingredient.

According to an aspect of some embodiments of the present invention there is provided a kit for inhibiting the coagulation of blood, comprising an effective amount of medical grade, disinfected aragonite.

According to some embodiments of the invention the calcium carbonate comprises coral exoskeleton from a coral of the Porites species.

According to some embodiments of the invention the coral is Porites lutea.

According to some embodiments of the invention the calcium carbonate, aragonite or coral exoskeleton is particulate calcium carbonate, aragonite or coral exoskeleton.

According to some embodiments of the invention the particulate calcium carbonate or aragonite or coral skeleton has an average particle diameter of between about 1 micrometer and about 3 mm.

According to some embodiments of the invention the particulate calcium carbonate, aragonite or coral exoskeleton comprises particles have an average particle diameter in the range of 0.1-10, 0.25-8.0, 0.5-5.0, 0.8-2.5, 1.0-3.0, or 0.05-0.5, 0.5-0.75, 0.75-1.0, 1.0-2.0, 2.0-5.0, 3.0-10.0 millimeters.

According to some embodiments of the invention the particulate calcium carbonate, aragonite or coral exoskeleton comprises particles have an average particle diameter of at least 0.5, at least 0.75, at least 1.0, at least 1.25, at least 1.5 or at least 2.0 millimeters.

According to some embodiments of the invention the particulate calcium carbonate, aragonite or coral exoskeleton comprises particles have an average particle diameter in the range of 0.5 micrometers to 10 millimeter, 1.0 micrometer to 100 micrometers, 2.0 micrometers to 75 micrometers, 5.0 micrometer to 50 micrometers, 7.5 micrometer to 30 micrometers, 5.0 micrometer to 20 micrometers, 50 micrometers to 150 micrometers and 1.0 micrometer to 10.0 micrometers.

According to some embodiments of the invention the particulate calcium carbonate, aragonite or coral exoskeleton comprises particles have an average particle diameter of at least 0.5, at least 0.75, at least 1.0, at least 2, at least 3, at least 5, at least 7.0, at least 10.0, at least 12.5, at least 15, at least 20, at least 35, at least 50, at least 75, at least 100 or at least 250 micrometers.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A and 1B are reproductions of photographs of murine blood either contacted or not contacted with coral exoskeleton (CS) in accordance with an embodiment of the teachings herein showing inhibition of in-vitro coagulation with addition of coral exoskeleton (CS);

FIGS. 2A-2F are reproductions of photographs of injured adult mice brains with and without contact of coral exoskeleton in accordance with an embodiment of the teachings herein, showing in-vivo inhibition of coagulation in brain wounds, with addition of coral exoskeleton (CS); and

FIGS. 3A-3C are reproductions of photographs of injured adult mice brains with and without contact of coral exoskeleton in accordance with an embodiment of the teachings herein, showing in-vivo thrombolytic activity of coral skeleton (CS) in brain wounds, with addition of coral exoskeleton (CS).

### DESCRIPTION OF SPECIFIC EMBODIMENTS OF THE INVENTION

Some embodiments of the invention relate to methods, compositions and devices suitable for influencing the coagulation of blood, especially mammalian blood, for example anticoagulants for reducing the rate of clotting of blood and decoagulants suitable for assisting in a process of dissolution of clotted blood.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Administration of anticoagulant compositions (e.g. tissue plasminogen activator, Streptokinase, Urokinase) for thrombolysis or prevention of clotting incurs a serious risk of bleeding complications, often in organs and tissues remote from, and unrelated to the site or origin of the blood clot. Thus, alternative solutions effective for preventing or dissolving blood clots, while minimizing the risks of collateral pathological bleeding, are needed. The present inventors have surprisingly shown that contacting blood with preparations of crystalline calcium carbonate, in the form of coral exoskeleton inhibits blood coagulation and is also effective in causing lysis (decoagulation) of already formed blood clots. Coral exoskeleton administration was effective as an anti-coagulant in both in-vitro (see Examples 1 and 2, herein) and live animal models (see Examples 3 and 4, herein).

Thus, according to one aspect of the present invention there is provided a method for influencing coagulation of blood comprising contacting an effective amount of calcium carbonate with blood, thereby inhibiting coagulation of the blood and/or dissolving clotted blood.

The term “calcium carbonate” as used herein, refers to the chemical compound CaCO₃. In some embodiments, the calcium carbonate is solid calcium carbonate, which can be in crystalline or amorphous form. As used herein, crystalline forms of calcium carbonate include aragonite, calcite, ikaite, vaterite and monohydrocalcite. Other solid forms of calcium carbonate include amorphous calcium carbonate.

Calcium carbonate useful for the present invention can be obtained from natural sources, or prepared chemically. Natural sources of calcium carbonate include, but are not limited to rock formations, such as limestone, chalk, marble, travertine and tufa. Calcium carbonate is also a principle structural component of many life forms, and thus can be obtained from, inter alia, corals, plankton, coralline algae, sponges, brachiopods, echinoderms, bryozoa, mollusks and other calcium carbonate-containing organisms.

In some embodiments the calcium carbonate comprises aragonite. As used herein, the term “aragonite” refers to the crystalline form of calcium carbonate, which can be commonly found in as mineral deposits in caves and in oceans, and in the shells of mollusks and exoskeleton of cold and warm-water corals. In some embodiments, the calcium carbonate comprises calcite. As used herein, the term “calcite” refers to a crystalline form of calcium carbonate, differing from aragonite in its crystal lattice formation, which can be obtained from sedimentary rocks and from the shells of some marine organisms. In some embodiments, the calcium carbonate comprises both aragonite and calcite. In other embodiments, the calcium carbonate is selected from the group consisting of aragonite, calcite, ikaite, vaterite, monohydrocalcite and amorphous calcium carbonate. In another embodiment, the calcium carbonate comprises one or more forms of crystalline calcium carbonate selected from the group consisting of aragonite, calcite, ikaite, vaterite and monohydrocalcite.

In one embodiment, the aragonite comprises a coral exoskeleton. The term “coral exoskeleton”, as used herein, refers to the exoskeleton of marine madreporic corals or material derived therefrom. Natural coral (Porites) consists of a mineral phase, principally calcium carbonate in the structural form of aragonite or calcite with impurities, such as Sr, Mg and F ions, and an organic matrix. Thus, as used herein, “coral exoskeleton” includes calcium carbonate in the form of aragonite or calcite, with or without additional components (minerals, organic and inorganic components) derived from or secreted by the living coral or life forms associated therewith.

In one embodiment, the coral is Porites. Coral exoskeleton is also commercially available (e.g. Biocoral™) and has been reported to be biocompatible and resorbable. Coral-derived material described as coralline HA prepared by hydrothermally converting the original calcium carbonate of the coral Porites in the presence of ammonium phosphate, maintaining the original interconnected macroporosity of the coral, is also commercially-available (Pro Osteon®, Interpore Cross). The high content calcium carbonate coral exoskeleton has since been shown to be biocompatible and biodegradable at variable rates depending on porosity, the implantation site and the species.

In some embodiments of the invention, the coral exoskeleton or compositions comprising the same are derived from a coral. In one embodiment, the coral can comprise any species, including, but not limited to, Porites, Acropora, Millepora, or a combination thereof. In one embodiment, the coral is from the Porites species. In one embodiment, the coral is Porites Lutea. In another embodiment, the coral is from the Acropora species. In another embodiment, the coral is Acropora grandis (which in one embodiment is very common, fast growing, and easy to culture). Acropora samples can be easily collected in sheltered areas of the coral reefs and/or can conveniently be cultured.

In another embodiment, the coral is from the Millepora species. In one embodiment, the coral is Millepora dichotoma. In one embodiment, the coral has a pore size of 150 microns and can be cloned and cultured, making Millerpora useful in the compositions and methods of this invention.

In another embodiment, the coral can be from any one or more of the following species: Favites halicora; Goniastrea retiformis; Acanthastrea echinata; Acanthastrea hemprichi; Acanthastrea ishigakiensis; Acropora aspera; Acropora austera; Acropora sp. “brown digitate”; Acropora carduus; Acropora cerealis; Acropora chesterfieldensis; Acropora clathrata; Acropora cophodactyla; Acropora sp. “danai-like”; Acropora divaricata; Acropora donei; Acropora echinata; Acropora efflorescens; Acropora gemmifera; Acropora globiceps; Acropora granulosa; Acropora cf hemprichi; Acropora kosurini; Acropora cf loisettae; Acropora longicyathus; Acropora loripes; Acropora cf lutkeni; Acropora paniculata; Acropora proximalis; Acropora rudis; Acropora selago; Acropora solitaryensis; Acropora cf spicifera as per Veron; Acropora cf spicifera as per Wallace; Acropora tenuis; Acropora valenciennesi; Acropora vaughani; Acropora vermiculata; Astreopora gracilis; Astreopora myriophthalma; Astreopora randalli; Astreopora suggesta; Australomussa rowleyensis; Coscinaraea collumna; Coscinaraea crassa; Cynarina lacrymalis; Distichopora violacea; Echinophyllia echinata; Echinophyllia cf echinoporoides; Echinopora gemmacea; Echinopora hirsutissima; Euphyllia ancora; Euphyllia divisa; Euphyllia yaeyamensis; Favia rotundata; Favia truncatus; Favites acuticollis; Favities pentagona; Fungia granulosa; Fungia klunzingeri; Fungia mollucensis; Galaxea acrhelia; Goniastrea edwardsi; Goniastea minuta; Hydnophora pilosa; Leptoseris explanata; Leptoseris incrustans; Leptoseris mycetoseroides; Leptoseris scabra; Leptoseris yabei; Lithophyllon undulatum; Lobophyllia hemprichii; Merulina scabricula; Millepora dichotoma; Millepora exaesa; Millipora intricata; Millepora murrayensis; Millipore platyphylla; Monastrea curta; Monastrea colemani; Montipora caliculata; Montipora capitata; Montipora foveolata; Montipora meandrina; Montipora tuberculosa; Montipora cf vietnamensis; Oulophyllia laevis; Oxypora crassispinosa; Oxypora lacera; Pavona bipartita; Pavona venosa; Pectinia alcicornis; Pectinia paeonea; Platygyra acuta; Platygyra pini; Platygyra sp “green”; Platygyra verweyi; Podabacia cf lanakensis; Porites annae; Porites cylindrica; Porites evermanni; Porites monticulosa; Psammocora digitata; Psammocora explanulata; Psammocora haimeana; Psammocora superficialis; Sandalolitha dentata; Seriatopora caliendrum; Stylocoeniella armata; Stylocoeniella guentheri; Stylaster sp.; Tubipora musica; Turbinaria stellulata; or any coral known in the art, or a combination thereof.

In yet another embodiment, coral for use in compositions or methods of this invention include, but are not limited to Madreporaria, Helioporida of the order Coenothecalia, Tubipora of the order Stolonifera, Millepora of the order Milleporina, or others known in the art. In some embodiments, coral for use in the compositions and methods of this invention may comprise scleractinian coral, including in some embodiments, Goniopora and others. In some embodiments, coral for use in the compositions and methods of this invention may comprise Alveoppora or bamboo corals, including in some embodiments, coral from the family Isididae, genera Keratoisis, Isidella, and others.

Coral exoskeleton is porous. In one embodiment, the average pore size (diameter) of a coral suitable for use in the compositions or methods of the invention is in the range of 1 micron-1 millimeter. In one embodiment, the average pore size of a coral is 30-180 microns. In another embodiment, the average pore size of a coral is 50-500 microns. In another embodiment, the average pore size of a coral is 150-220 microns. In one embodiment, the average pore size of a coral is 250-1000 microns.

Aragonite suitable for use in compositions and/or methods of the invention may be prepared from coral or coral fragments, or from coral sand. Briefly, the coral can be prepared as follows: in one embodiment, coral or coral sand is purified from organic residues, washed, bleached, frozen, dried, sterilized or a combination thereof prior to use in the compositions and/or methods of the invention.

The calcium carbonate, aragonite, or coral exoskeleton of the invention can be provided in a variety of forms, shapes and structures, compatible with many different applications of the invention. Some suitable forms and shapes can include, but are not limited to, for example, layers, blocks, spherical and hollow spherical forms, concentric spheres, rods, sheets, symmetrical and asymmetrical forms, amorphous and other irregular shapes and particles. The calcium carbonate, aragonite or coral exoskeleton can be shaped, for example, to fit a particular cavity or surface of tissue, or to fit the contours of a device. In some embodiments, the calcium carbonate, aragonite or coral exoskeleton is provided as particulate calcium carbonate, aragonite or coral exoskeleton.

In some embodiments, preparation of the aragonite or coral exoskeleton includes contacting solid aragonite (e.g coral exoskeleton) of a desired size and shape with a solution comprising an oxidizing agent, and washing and drying the solid aragonite.

In some embodiments, the oxidizing agent for use in the processes of this invention may be any suitable oxidizing agent, which facilitates the removal of organic debris from the coral exoskeletons. In some embodiments, the oxidizing agent is sodium hypochlorite.

For most therapeutic applications, it is desirable that the calcium carbonate, or aragonite, when derived from natural sources, such as coral, be devoid of any cellular debris or other organisms associated therewith in its natural state. Thus, in some embodiments, the coral exoskeleton is an acellular coral exoskeleton.

According to this aspect, and in some embodiments, the process comprises conducting said contacting under mildly acidic conditions.

In one embodiment, calcium carbonate, aragonite or coral suitable for use in the compositions and/or methods of the invention is produced from coral or coral sand according to a process comprising washing ground solid calcium carbonate (e.g. aragonite), such as coral or naturally occurring coral sand with water to desalinate it, then disinfecting and drying the desalinated coral sand at temperatures of about 80 degrees to about 150 degrees C., preferably 90 degrees to 120 degrees C., cutting larger pieces of coral into small pieces, and grinding the disinfected and dried coral or coral sand into small particles, including but not limited to particles of 1-10 microns. In some embodiments, coral is ground into particles having a particle diameter of in the range of 1-5, 1-20, 1-50, 1-100, 5-10, 10-15, 15-20, 10-50, 10-100, 20-100, 50-100, 80-150, 100-200, 100-350 or 150-500 microns across, and a particle volume in the range of 1-100, 50-500, 250-1000, 500-2500, 1000-5000 and 2500-10,000 cubic micron to 0.01-0.1, 0.05-0.5, 0.5-0.75, 0.75-1.0, 1.0-2.0 and 1.0-5.0 cubic millimeters in volume.

As used herein, the term “coagulation of blood” refers to clot formation in blood resulting from either or both of the intrinsic cascade, initiated when contact is made between blood and exposed negatively charged surfaces, and the extrinsic pathway, initiated upon vascular injury, leading to activation of factor X to Xa which hydrolyzes and activates prothrombin to thrombin. Thrombin then activates factors XI, VIII and V, until ultimately fibrinogen is converted to fibrin and factor XIII to XIIIa. Factor XIIIa (also termed transglutaminase) cross-links fibrin polymers solidifying the clot. Thus, as used herein, the term “clot” or “thrombus” refers to the final product of the blood coagulation step in hemostasis. There are two components to a clot/thrombus: aggregated platelets that form a platelet plug, and a mesh of cross-linked fibrin protein. The substance making up a thrombus is also known as cruor.

Thrombin-clottable fibrinogen is found in all vertebrate animals, but not in protochordates (amphioxus, tunicates, etc.) or invertebrate animals. The earliest diverging vertebrates (lampreys and hagfish) have six-chained, fully differentiated fibrinogens that polymerize and cross-link the same as mammalian ones, and it is well established that vitamin-K dependent factors play a role in the clotting of lower vertebrates like the lamprey and hagfish. Thus, the coral exoskeleton and compositions comprising the same can be used to influence (e.g. reduce) coagulation of both mammalian and non-mammalian blood. In some embodiments of the methods and compositions of the present invention, the blood is mammalian blood. In some embodiments, the blood is human blood.

With respect to “coagulation of blood”, the calcium carbonate, aragonite or coral exoskeleton and compositions comprising the same of the present invention influence coagulation and influence the coagulation state of blood (e.g, reduce or inhibit coagulation or coagulation state of the blood). As such, the calcium carbonate, aragonite or coral exoskeleton and compositions comprising the same are characterized by influencing (e.g. inhibiting) the clotting of blood and blood clotting state, which includes the clotting of plasma, as well as enhancing or increasing lysis or dissolving of a blood clot. Thus, according to some embodiments of the invention, the coral exoskeleton or compositions comprising the same are contacted with at least some unclotted blood, the contacting influencing (e.g. inhibiting) coagulation of the unclotted blood. According to other embodiments of the invention, the calcium carbonate, aragonite or coral exoskeleton or compositions comprising the same are contacted with at least some clotted blood, the contacting at least partially dissolving the clotted blood.

Clotting of blood can be monitored in a variety of assays. In one aspect, the coral exoskeleton or composition comprising same of the present invention increases the clotting time of human plasma as measured in both the prothrombin time (PT) and activated partial thromboplastin time (aPTT) assays.

In the PT assay, clotting is initiated by the addition of a fixed amount of tissue factor-phospholipid micelle complex (thromboplastin) to human plasma. Compositions capable of reducing coagulation (e.g. anticoagulants) interfere with certain interactions of this complex and increase the time required to achieve clotting relative to the clotting observed in the absence of the anticoagulant. Thus, the ability of calcium carbonate, aragonite or coral exoskeleton or composition comprising same to act as an inhibitor in this assay can be predictive of anticoagulant activity in vivo.

In the aPTT assay, clotting is initiated by the addition of a certain fixed amount of negatively charged phospholipid micelle (activator) to the human plasma. Substances, acting as anticoagulants will interfere with certain interactions of the complex and again increase the time to achieve a certain amount of clotting relative to that observed in the absence of the anticoagulant. Such coagulation assays are well known in the art, and are described in detail in, for example, US Patent Publication 20100240584 to Vlasuk et al. These assays can be used to assess anticoagulant activity of the calcium carbonate, aragonite or coral exoskeleton and compositions comprising the same of the present invention.

The calcium carbonate, aragonite or coral exoskeleton and compositions of the present invention include those which can double the clotting time of human plasma in the PT assay and which can also double the clotting time of human plasma in the aPTT assay, when provided in ratio of 0.5-150 gram/liter of plasma. In some embodiments the calcium carbonate, aragonite or coral exoskeleton and compositions comprising calcium carbonate, aragonite or coral exoskeleton can double the clotting time of human plasma in the PT assay and which can also double the clotting time of human plasma in the aPTT assay, when provided in ratio of 0.5-150 gram/liter of plasma.

In some embodiments, the calcium carbonate, aragonite or coral exoskeleton is provided at a ratio of 0.5-150 gram/liter, 1.0-120 gram/liter, 2.0-110 gram/liter, 5-100 gram/liter, 8-100 gram/liter, 10-100 gram/liter, 20-90 gram/liter, 30-80 gram/liter, 40-120 gram/liter, 50-150 gram/liter, 60-100 gram/liter, 70-120 gram/liter, 50-150 gram/liter, or 50-120 gram/liter of plasma. In particular embodiments, the calcium carbonate, aragonite or coral exoskeleton is provided at a ratio of 40 grams/liter, 50 grams/liter, 55 grams/liter, 60 grams/liter, 70 grams/liter, 80 grams/liter, 90 grams/liter, 100 grams/liter, 120 grams/liter or 150 grams/liter of plasma.

Anticoagulant activity of the calcium carbonate, aragonite or coral exoskeleton and compositions comprising the same of the present invention can also be evaluated ex-vivo in a simple mammalian (e.g murine, human) blood coagulation assay—whole mammalian blood, or fluid containing whole mammalian blood is drawn fresh into a tube with or without the calcium carbonate, aragonite or coral exoskeleton or composition comprising the same, and time to clotting is recorded (clotting can be determined optically or otherwise). Detailed description of such an assay is provided in Example 1, herein.

Anticoagulant, or antithrombotic, activity of calcium carbonate, aragonite or coral exoskeleton and compositions comprising the same of the present invention also can be evaluated using the in vivo models presented in Examples 3 and 4. The murine model described in Example 3 is a model of platelet dependent thrombosis that is commonly used to assess antithrombotic compounds. The model evaluates the ability of a test compound to prevent the formation of a thrombus induced by mechanical trauma in a segment of the mouse brain (cortical hemisphere), by application of the test compound (e.g. coral exoskeleton particles) simultaneous with the wounding of the tissue, and assessment of the extent of clotting in histological sections at a pre-determined time or times post-trauma. Calcium carbonate, aragonite or coral exoskeleton or compositions comprising the same of the present invention are effective anticoagulants in this model when administered at the time of wounding.

One desired effect of an anticoagulant is that it inhibits blood coagulation, or thrombus formation, and/or increases thrombolysis, or blood clot dissolution or decoagulation. The murine model described in Example 4 is a model of decoagulation that is used to assess thrombolytic compounds. The model evaluates the ability of a test compound to enhance the dissolution of a thrombus induced by mechanical trauma in a segment of the mouse brain (cortex), by application of the test compound (e.g. coral exoskeleton particles) at a given time following the wounding of the tissue, and assessment of the extent of clotting in histological sections at a pre-determined time or times thereafter. Calcium carbonate, aragonite or coral exoskeleton or compositions comprising the same of the present invention are effective decoagulants in this model when administered following wounding of the tissue.

Thus, according to some aspects of the invention, at least some of the blood is clotted blood, and contacting the calcium carbonate at least partially dissolves the clotted blood.

As used herein, the term “dissolves the clotted blood” refers to partial or complete conversion of at least a portion of the coagulated, gel-like clot to a plurality of soluble, non-precipitated components which can be borne by blood or lymph. “Dissolving clotted blood” also refers to “de-coagulation” or “thrombolysis” or “clot busting”, in which the process of fibrinolysis reduces the fibrin matrix comprising the blood clot, and can release any clot component restricted (inside or outside) by the fibrin clot. Partial decoagulation or clot dissolution refers to a state in which only a portion of the clot undergoes weakening and eventual lysis of the fibrin network of the clot.

Calcium carbonate, aragonite or coral exoskeleton, compositions and devices comprising the same of the present invention are useful as potent inhibitors of blood coagulation in vitro, ex-vivo and in vivo. As such, they are useful as in vitro and ex-vivo reagents to prevent the clotting of blood and enhance decoagulation, and are also useful as in vivo agents to prevent or inhibit thrombosis or blood coagulation, as well as enhance decoagulation (thrombolysis) in animals.

Thus, according to some embodiments of the present invention, contacting the calcium carbonate, aragonite or coral exoskeleton or compositions comprising the same with the blood comprises applying calcium carbonate, aragonite or coral exoskeleton to a wound. In some embodiments, applying the calcium carbonate, aragonite or coral exoskeleton to the wound can be effected by direct application or injection.

According to other embodiments of the present invention, contacting the calcium carbonate, aragonite or coral exoskeleton or compositions comprising the same with blood is effected in an ischemic tissue or a region of tissue at risk of ischemia.

As used herein, the term “ischemia” refers to the state of reduced, abnormally decreased circulation, resulting in decreased provision of blood-borne components (e.g. nutrients, gasses, co-factors, hormones, etc) and decreased elimination of wastes (e.g. respiratory waste gasses, metabolic wastes and by-products). Ischemia can be the result of partial reduction in circulation, or the result of complete failure of circulation to a tissue or portion thereof. Ischemia can stimulate specific cellular responses in some tissues, such as adaptations to reduced oxygen tension and nutrients, and prolonged ischemia can result in more severe responses, such as apoptosis and/or cell death. Ischemia can lead to tissue necrosis and gangrene. Tissue at risk of ischemia, as used herein, is a tissue experiencing, or at risk of experiencing, a reduced circulation (as a result of damage to a blood vessel, e.g. constriction or severing), reduced availability of blood, and at risk of experiencing the reduction or elimination of blood-borne components (e.g. nutrients, gasses, co-factors, hormones, etc).

In some embodiments, applying the calcium carbonate, aragonite or coral exoskeleton to the ischemic tissue or tissue, or portion thereof at risk of ischemia can be effected by direct application or injection.

In some embodiments, the calcium carbonate, aragonite or coral exoskeleton is provided as particulate calcium carbonate, aragonite or coral exoskeleton. The particles typically have a particle diameter in the range of 0.5 micrometers to 10 millimeter, 1.0 micrometer to 100 micrometers, 2.0 micrometers to 75 micrometers, 5.0 micrometer to 50 micrometers, 7.5 micrometer to 30 micrometers, 5.0 micrometer to 20 micrometers, 1.0 micrometer to 10.0 micrometers, 50.0 micrometers to 10 millimeters, 100.0 micrometers to 5 millimeters, 200.0 micrometers to 2.5 millimeters, 0.5 millimeter to 1.25 millimeters, and 0.5 millimeters to 1.0 millimeters. According to some embodiments of the invention, the particles typically have a particle diameter in the range of at least 0.5, at least 0.75, at least 1.0, at least 2, at least 3, at least 5, at least 7.0, at least 10.0, at least 12.5, at least 15, at least 20, at least 35, at least 50, at least 75, at least 100, at least 250, at least 500 micrometers, at least 0.75 millimeters, at least 1.0 millimeters, at least 1.2 millimeters, at least about 1.3 millimeters, at least about 1.5 millimeters, at least about 2.0 millimeters, at least about 2.5 millimeters, at least about 3 millimeters, at least about 4 millimeters, at least about 5 millimeters, at least about 6 millimeters, at least about 7 millimeters, at least about 8 millimeters, at least about 9.0 millimeters, at least about 10 millimeters, and all effective intermediate particle diameters are contemplated. In one embodiment, the particles have a particle diameter of 0.5-20 micrometers, 1-18 micrometers, 1.5-15 micrometers, 2-17 micrometers, 3-12 micrometers, 4-10 micrometers, 5-8 micrometers, 5-6 micrometers and 10-20 micrometers.

The calcium carbonate, aragonite or coral exoskeleton can be administered (applied) as relatively large particles, for example, directly implanted, for example, by a forceps or topically (e.g. sprinkling) into or onto a region of a large blood clot(s) in order to inhibit further clotting, dissolve the clot and restore circulation. Thus, in some embodiments, the particles are at least about 0.1-10, 0.25-8.0, 0.5-5.0, 0.8-2.5, 1.0-3.0, or 0.05-0.5, 0.5-0.75, 0.75-1.0, 1.0-2.0, 2.0-5.0 millimeters in particle diameter and at least about 0.05-0.5, 0.5-0.75, 0.75-1.0, 1.0-2.0 or at least 1.0-5.0 cubic millimeters in volume. In some embodiments, the particles have a particle diameter of at least 0.5, at least 0.75, at least 1.0, at least 1.25, at least 1.5 or at least 2.0 millimeters. The calcium carbonate, aragonite or coral exoskeleton can also be administered (applied) as small sized particles, in the micrometer range, suitable for topical administration but also for administration by injection, for example, injection into a blood vessel, duct or other passage or tube-like structure where direct or topical administration is inconvenient, contra-indicated or otherwise undesirable. Administration (application) of small sized particles is envisaged for, for example, but not limited to injection into and proximally to blood clot(s) and thrombi in occluded blood vessels. Thus, in some embodiments, the calcium carbonate, aragonite or coral exoskeleton particles are at least 1-5, 5-10, 10-50, 75-100, 100-500, 250-1000 or at least 1000-10000 cubic micrometers in volume. In some embodiments, the particles are 0.5, 0.75, 1.0, 1.25, 1.5, 2.0, 3.0, 3.5, 4.0, 4.5, 5.0, 6.0 or 8.0-10 cubic micrometers in volume. In some embodiments, the coral exoskeleton particles have a particle diameter of at least 0.5, at least 0.75, at least 1.0, at least 1.25, at least 1.5, at least 2.0, at least 3.0, at least 3.5, at least 4.0, at least 4.5, at least 5.0, at least 6.0 or 8.0-10 micrometers. In one embodiment, the particles have a particle diameter of 0.5-20 micrometers, 1-18 micrometers, 1.5-15 micrometers, 2-17 micrometers, 3-12 micrometers, 4-10 micrometers, 5-8 micrometers, 5-6 micrometers and particularly 10-20, 5-15 or 12-18 micrometers.

In some embodiments, the coral exoskeleton is a particulate coral exoskeleton which maintains the structure of coral exoskeleton. Coral exoskeleton commonly comprises porous sponge-like elements including, inter-alia, the septo-costae and coenosteum. Thus, in some embodiments, the particulate coral exoskeleton maintains a porous exoskeleton structure.

It will be appreciated that vascular (e.g. injection into a blood vessel) administration (application) of the calcium carbonate, aragonite or coral exoskeleton particles or compositions of the invention, while affording access to otherwise unexposed surfaces, bears the risk of the particles being released to other, remote and un-targeted regions of the subject's body, via circulation of fluid (e.g. blood) in the vessel. However, the calcium carbonate, aragonite or coral exoskeleton is biodegradable, yielding a calcium salt, carbon dioxide and water in an acidic environment, and thus, the dangers of undesirable side effects of such administration of minute coral exoskeleton particles are minimized.

Suitable routes of administration of the calcium carbonate, aragonite or coral exoskeleton or compositions comprising the same can, for example, include intramuscular, subcutaneous and intramedullary injections as well as intrathecal, direct intraventricular, intracardiac, e.g., into the right or left ventricular cavity, into the common coronary artery, intravenous or intraperitoneal injections.

Conventional approaches for delivery to the central nervous system (CNS) include neurosurgical strategies (e.g., intracerebral injection or intracerebroventricular infusion).

One may administer the calcium carbonate, aragonite or coral exoskeleton or composition comprising same in a direct, local manner, for example, via placement or injection of the pharmaceutical composition directly into a tissue region of a patient. Thus, according to one embodiment of the invention, contacting the coral exoskeleton with blood comprises applying particulate calcium carbonate, aragonite or coral exoskeleton to tissue.

The term “tissue” refers to part of an organism consisting of cells designed to perform a function or functions. Examples of tissue include, but are not limited to, brain tissue, retina, skin tissue, hepatic tissue, pancreatic tissue, bone, cartilage, connective tissue, blood tissue, muscle tissue, cardiac tissue, gastrointestinal tissue, vascular tissue, renal tissue, pulmonary tissue, gonadal tissue, hematopoietic tissue.

According to some embodiments, the calcium carbonate, aragonite or coral exoskeleton and compositions comprising the same of the present invention are also useful as pharmaceutical agents for preventing or inhibiting thrombosis or blood coagulation in an animal. This prevention or inhibition of thrombosis or blood coagulation includes preventing or inhibiting abnormal thrombosis and decoagulation (thrombolysis) of existing clots.

Conditions characterized by abnormal thrombosis are well known in the medical arts and include those involving the arterial and venous vasculature of mammals. With respect to the coronary arterial vasculature, abnormal thrombosis (thrombus formation) characterizes the rupture of an established atherosclerotic plaque which is the major cause of acute myocardial infarction and unstable angina, and also characterizes the occlusive coronary thrombus formation resulting from either thrombolytic therapy or percutaneous transluminal coronary angioplasty (PTCA). With respect to the venous vasculature, abnormal thrombosis characterizes the condition observed in patients undergoing major surgery in the lower extremities or the abdominal area who often suffer from thrombus formation in the venous vasculature resulting in reduced blood flow to the affected extremity and a predisposition for pulmonary embolism. Abnormal thrombosis further characterizes disseminated intravascular coagulopathy which commonly occurs within both vascular systems during septic shock, certain viral infections and cancer, a condition wherein there is rapid consumption of coagulation factors and systemic coagulation which results in the formation of life-threatening thrombi occurring throughout the microvasculature leading to widespread organ failure.

In the case of ischemic cerebral vascular accident (“stroke”) associated with clot formation in the affected portion of the brain, application of the calcium carbonate, aragonite or coral exoskeleton and compositions comprising the same, alone or along with anti-coagulant drugs can be effective in lysing the occluding embolus and reducing further clotting. Application of the calcium carbonate, aragonite or coral exoskeleton and compositions comprising the same in the brain can be performed by direct administration, e.g. topically applying the calcium carbonate, aragonite or exoskeleton and compositions to the clot, or by intravenous/intraarterial injection of the coral exoskeleton and compositions comprising the same at or near to the occluding embolus. Other means of delivering the calcium carbonate, aragonite or coral exoskeleton and compositions comprising the same intravascularly to the clot can also be employed (e.g. cannulae).

In some embodiments, the composition, uses and methods of treatment are implemented for treating nervous tissue such as the brain or spine. In such embodiments, the nervous tissue is any suitable nervous tissue.

In some embodiments, the nervous tissue is selected from the group consisting of: nervous tissue of an embryonic organism; nervous tissue of a fetal organism, nervous tissue of a newborn (in humans, up to 28 days old), nervous tissue of an infant (in humans from 29 days to 1 year old; nervous tissue of a young organism (in humans, about 1 to about 9 years); nervous tissue of an adolescent organism (in humans about 9 to about 14 years); nervous tissue of a young adult organism (in humans, about 15 to about 30 years); nervous tissue of an adult organism (in humans about 30 to about 70 years); and nervous tissue of an aged organism (in humans, about 70 years and above).

As reported in a number of scientific publications, (e.g., Shany et al; Tissue Engineering 2006, 12(7), 1763-1773: Peretz et al., Tissue Engineering 2007, 13(3), 461-472), the Inventor and coworkers implanted aragonite coral exoskeleton particles (0.5-1 mm³) in the cortical region of the brain of 1-2 day old rat pups (equivalent to human newborns). Two weeks after implantation in the living pups, it was observed that the implanted coral exoskeleton was invaded by astrocytes and neurons. These results suggested potential therapeutic uses for treatment of nerve tissue, also in older organisms.

The calcium carbonate, aragonite or coral exoskeleton and compositions comprising the same of the present invention are useful as in vitro reagents for inhibiting clotting in blood drawing tubes, blood collection bags and blood storage vessels (bags, flasks, tubes, etc). For example, the use of stoppered test tubes having a vacuum therein as a means to draw blood obtained by venipuncture into the tube is well known in the medical arts. Kasten, B. L., “Specimen Collection”, Laboratory Test Handbook, 2nd Edition, Lexi-Comp Inc., Cleveland pp. 16-17 (Edits. Jacobs, D. S. et al. 1990). Such vacuum tubes may be free of clot-inhibiting additives, in which case, they are useful for the isolation of mammalian serum from the blood. They may alternatively contain clot-inhibiting additives (such as heparin salts, EDTA salts, citrate salts or oxalate salts), in which case, they are useful for the isolation of mammalian plasma from the blood. The calcium carbonate, aragonite or coral skeleton and compositions comprising the same of the present invention are potent inhibitors of blood clotting and potent decoagulants, and as such, can be incorporated into blood collection and storage tubes to prevent clotting of the mammalian blood drawn into them.

The calcium carbonate, aragonite or coral exoskeleton and compositions comprising the same of the present invention are used alone, or in combination with other known inhibitors of clotting, in the blood collection tubes, for example, with heparin salts, EDTA salts, citrate salts or oxalate salts.

The amount to be added to such tubes, or effective amount, is that amount sufficient to influence (e.g. inhibit) the formation of a blood clot when mammalian blood is drawn into the tube or vessel. In some embodiments, the calcium carbonate, aragonite or coral exoskeleton and compositions comprising the same of the present invention are added to blood collection tubes in such amounts that, when combined with, for example, 2 to 10 ml of mammalian blood, the amount of such calcium carbonate, aragonite or coral exoskeleton and compositions comprising the same will be sufficient to inhibit the formation of blood clots. Typically, this effective amount is that required to give a final ratio of 1-250, 5-225, 10-200, 20-175, 25-150, 50-150, 60-140, 70-130, 80-120 and 90-100 grams/liter of blood, particularly 25-150, 35-80, 45-75 and 50-60 grams/liter of blood.

Calcium carbonate, aragonite or coral exoskeleton can be used to coat surfaces, for example, of a device such as a medical device, in order to reduce coagulation and enhance thrombolysis upon contact of the device with blood or a fluid comprising blood. Thus, according to some aspects of the invention, the calcium carbonate, aragonite or coral exoskeleton is comprised in a device.

The calcium carbonate, aragonite or coral exoskeleton can be used as a coating, adhered to a surface, e.g, the surface of a device, by adhesives, such as medically acceptable bioadhesives, polymer glues, etc., and can be applied to the device by dip coating with an adhesive base. Such dip coating can be effected during manufacture of the device, or at any time prior to implantation, including immediately prior to implantation. In some embodiments, the calcium carbonate, aragonite or coral exoskeleton, being a rigid, calcium carbonate crystalline structure, can be embedded within the material of the device, for example, embedded into the surface of a polymer by application of heat, or fused to glass surfaces of the device. In yet other embodiments, the calcium carbonate, aragonite or coral exoskeleton, or particulate calcium carbonate, aragonite or coral exoskeleton can be incorporated into the base material forming the device, for example, mixed within the components of a polymer (such as, but not limited to acrylic) before polymerization.

In some embodiments, the device can be an implantable medical device. The device comprising calcium carbonate, aragonite or coral exoskeleton can be any medical or other device which, within the course of use, contacts blood or a fluid comprising blood. Thus, the device can be any implantable medical device, such as artificial joints, artificial blood vessels, stents, cochlear implants, pacemakers, implantable defibrillators, bone screws and plates, coronary stents, blood vessel bypass, heart valve, blood vessel graft, vascular filters, an artificial heart, a shunt, an embolic protection device (such as distal or proximal occlusion aspiration devices, distal embolic filters, etc), a catheter, a cannula and other implantable medical devices. When the device is implanted into a patient's body, the blood compatibility of the calcium carbonate, aragonite or coral exoskeleton can reduce the occurrence or severity of adverse biological reactions, such as inflammation and/or formation of blood clots, associated with implantation of the device.

In some embodiments, the medical devices that contact blood are devices that contact blood outside of the body. Thus, in some embodiments, the contacting of the calcium carbonate, aragonite or coral exoskeleton with the blood or fluid comprising blood is effected ex-vivo. For instance, the calcium carbonate, aragonite or coral exoskeleton can be incorporated into the surface (coated) or into the base material of renal dialysis equipment, artificial (ex-corporeal) heart devices, blood donation and transfusion equipment, and other medical devices that handle, e.g., contain or transfer, blood outside of the body. The anti-coagulant capability of the calcium carbonate, aragonite or coral exoskeleton can reduce the occurrence or severity of blood clots or other adverse reactions in the blood handled by the medical devices.

In still other embodiments, the devices that contact blood are devices that contact blood in-vitro. Thus, in some embodiments, the contacting of the calcium carbonate, aragonite or coral exoskeleton with the blood or fluid comprising blood is effected in-vitro. For instance, the calcium carbonate, aragonite or coral exoskeleton can be incorporated into the surface (coated) or into the base material of laboratory or hematology equipment such as blood collection or storage devices, a conduit, a flask, a bottle, a dish, a petri dish, a plate, a multi-well plate, a test tube, a blood transfusion bag, and other devices that are designed to contact blood or fluids comprising blood in-vitro. Thus, according to some embodiments of the invention, there is provided a device for contacting blood comprising calcium carbonate, aragonite or coral exoskeleton. The device can be configured for placement within the body of a living animal, contacting the blood in-vivo, or can be configured for contacting the blood or fluid comprising blood ex-vivo or in-vitro.

Pharmaceutical compositions comprising calcium carbonate, aragonite or coral exoskeleton are envisaged in the present invention. Thus, according to some embodiments of the present invention, there is provided a pharmaceutical composition for inhibiting the coagulation of blood and/or dissolving clotted blood comprising particulate calcium carbonate, aragonite or coral exoskeleton and a pharmaceutically acceptable carrier.

Compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the composition may be formulated in aqueous solutions, preferably in physiologically compatible buffers such as Hank's solution, Ringer's solution, or physiological salt buffer.

The calcium carbonate, aragonite or coral exoskeleton or compositions comprising same described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, and may contain formulatory agents such as suspending, stabilizing and/or dispersing agents.

Alternatively, the calcium carbonate, aragonite or coral exoskeleton or composition comprising same may be in dry particulate form for constitution with a suitable vehicle, e.g., sterile, pyrogen-free water based solution, before use.

Compositions suitable for use in context of some embodiments of the invention include compositions wherein the calcium carbonate, aragonite or coral exoskeleton particles are contained in an amount effective to achieve the intended purpose. More specifically, a therapeutically effective amount means an amount of active ingredients (e.g. aragonite, coral exoskeleton) effective to prevent, alleviate or ameliorate symptoms of a disorder (e.g., coagulation) or prolong the survival of the subject being treated.

Determination of a therapeutically effective amount is well within the capability of those skilled in the art, especially in light of the detailed disclosure provided herein.

For any preparation or composition used in the methods of the invention, the therapeutically effective amount or dose can be estimated initially from in vitro assays. For example, a dose can be formulated in animal models to achieve a desired degree of inhibition of coagulation, or to achieve a desired degree of thrombolysis (complete or partial). Such information can be used to more accurately determine useful doses in humans.

Toxicity and therapeutic efficacy of the calcium carbonate, aragonite or coral exoskeleton particles and compositions comprising the same described herein can be determined by standard laboratory procedures in vitro or experimental animals. The data obtained from these in vitro and animal studies can be used in formulating a range of dosage for use in humans. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Depending on the severity and responsiveness of the condition to be treated, dosing can be of a single or a plurality of administrations, with course of treatment lasting from several days to several weeks or until cure is effected or diminution of the disease state (e.g. blood clot, thrombotic occlusion) is achieved.

The amount of coral exoskeleton or composition comprising the same to be administered will, of course, be dependent on the subject being treated, the severity of the affliction, the manner of administration, the judgment of the prescribing physician, etc.

Calcium carbonate, aragonite or coral exoskeleton or compositions comprising the same of some embodiments of the invention may, if desired, be presented in a pack or dispenser device, such as an FDA approved kit, which may contain one or more unit dosage forms containing the calcium carbonate, aragonite or coral exoskeleton or compositions comprising the same. The pack may, for example, comprise metal or plastic foil, such as a blister pack. The pack or dispenser device may be accompanied by instructions for administration. The pack or dispenser may also be accompanied by a notice associated with the container in a form prescribed by a governmental agency regulating the manufacture, use or sale of therapeutic compositions, which notice is reflective of approval by the agency of the form of the compositions or human or veterinary administration. Such notice, for example, may be of labeling approved by the U.S. Food and Drug Administration for therapeutic compositions or of an approved product insert. Compositions comprising a preparation of the invention formulated in a compatible carrier may also be prepared, placed in an appropriate container, and labeled for treatment of an indicated condition, as is further detailed above. The pack or kit may also comprise additional agents useful in influencing blood coagulation and treating conditions associated with pathological or undesired coagulation or blood clots, and the like.

Some additional agents suitable for use with the calcium carbonate, aragonite or coral exoskeleton of the invention, and/or compositions comprising the same, and methods for its use, include, but are not limited to anti-coagulants such as heparin, LMWheparin, plasminogen activator, streptokinase and urokinase, antibiotics, and anti-platelet drugs such as dipyridamole.

The term “treating” refers to inhibiting, preventing or arresting the development of a pathology (disease, disorder or condition) and/or causing the reduction, remission, or regression of a pathology. Those of skill in the art will understand that various methodologies and assays can be used to assess the development of a pathology, and similarly, various methodologies and assays may be used to assess the reduction, remission or regression of a pathology.

As used herein, the term “preventing” refers to keeping a disease, disorder or condition from occurring in a subject who may be at risk for the disease, but has not yet been diagnosed as having the disease.

As used herein, the term “subject” includes mammals, preferably human beings at any age which suffer from the pathology. Preferably, this term encompasses individuals who are at risk to develop the pathology.

As used herein the phrase “treatment regimen” refers to a treatment plan that specifies the type of treatment, dosage, schedule and/or duration of a treatment provided to a subject in need thereof (e.g., a subject diagnosed with a pathology). The selected treatment regimen can be an aggressive one which is expected to result in the best clinical outcome (e.g., complete cure of the pathology) or a more moderate one which may relief symptoms of the pathology yet results in incomplete cure of the pathology. It will be appreciated that in certain cases the more aggressive treatment regimen may be associated with some discomfort to the subject or adverse side effects (e.g., a damage to healthy cells or tissue). The type of treatment can include a surgical intervention (e.g., removal of lesion, diseased cells, tissue, or organ), a cell replacement therapy, an administration of a therapeutic drug (e.g., receptor agonists, antagonists, hormones, chemotherapy agents) in a local or a systemic mode, an exposure to radiation therapy using an external source (e.g., external beam) and/or an internal source (e.g., brachytherapy) and/or any combination thereof. The dosage, schedule and duration of treatment can vary, depending on the severity of pathology and the selected type of treatment, and those of skills in the art are capable of adjusting the type of treatment with the dosage, schedule and duration of treatment.

It is expected that during the life of a patent maturing from this application many forms of calcium carbonate, aragonite or coral exoskeleton, compositions comprising the same, methods for their use and devices comprising the same will be developed and the scope of the term calcium carbonate, aragonite or coral exoskeleton and/or compositions comprising the same is intended to include all such new technologies a priori.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

Experimental Materials and Methods

Preparation of Coral Exoskeleton

Exoskeleton from the coral Porites lutea was cut into small pieces (between about 0.25 and about 1 cm₃) and bleached with 7% hypochlorite solution. Residual organic matter was removed by washing with 1M NaOH solution followed by 35% H₂O₂ solution. Particulate coral exoskeleton was obtained by manually grinding the pieces of exoskeleton using a marble mortar and pestle. Particulate coral exoskeleton can also be obtained using a grainer device. The particulate coral exoskeleton was autoclaved before usage.

### Example 1

### In Vitro Effect of Coral Exoskeleton on Blood Coagulation

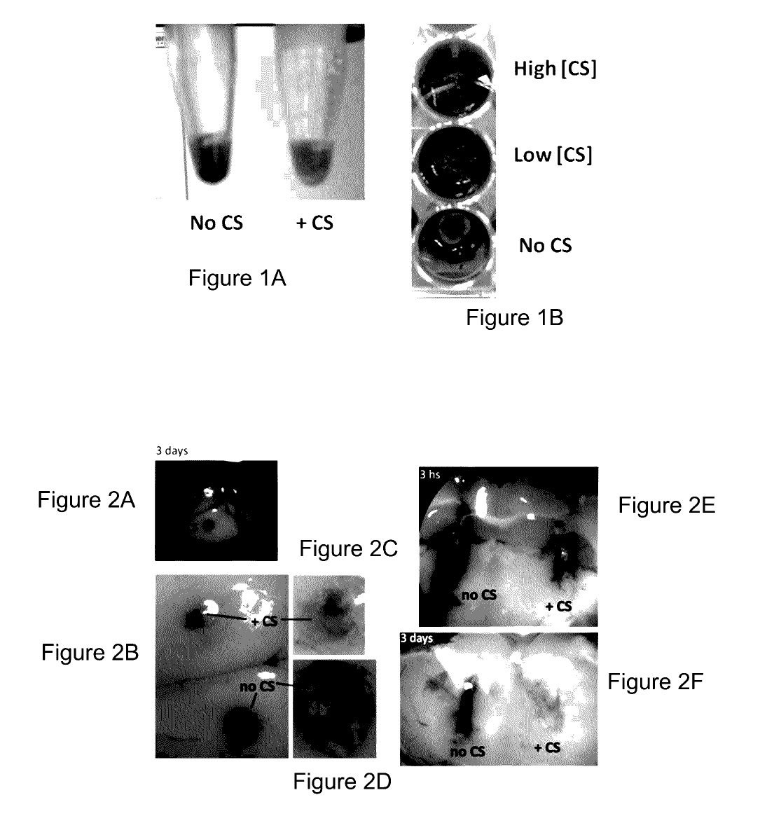

400 μl of blood drawn from adult mice was transferred to two tubes, one empty as a control and the second containing 40 mg of particulate coral exoskeleton. Within less than 5 minutes, blood clots appeared in the control, whereas no clots were observed in the tube containing the particulate coral exoskeleton, as shown in FIG. 1A.

Particulate coral exoskeleton was placed in two wells of a 96-well plate: 15 mg in a first well and 7.5 mg in a second well. 100 μl ml of freshly drawn mouse blood was placed in each of the two wells to contact the particulate coral exoskeleton as well as in a third well that was devoid of any particulate coral exoskeleton as a control. After 5 minutes, the appearance of blood clots was observed, in a dose-dependent manner (see FIG. 1B). Complete clotting was observed in the control well (“No CS”), a slight haze of clots was observed in the well containing 7.5 mg of coral exoskeleton (“Low [CS]”) and no clots were observed in the well containing 15 mg of coral exoskeleton (“High [CS]”).

### Example 2

### In Vitro Decoagulation of Blood by Coral Exoskeleton

400 μl of blood taken from adult mice was transferred to two empty tubes and left at room temperature until blood clots appeared. Subsequently, 40 mg particulate coral exoskeleton were added to one of the tubes and vortexed. To the second tube was added a PBS solution with glass beads as a control. 7 to 10 minutes after addition of the particles, the blood clots in the presence of the particulate coral exoskeleton became softer, reduced in size and in some experiments disappeared completely. In the control tube, the blood clots increased in size and eventually settled at the bottom of the tube.

### Example 3

### In Vivo Anti-Coagulation Effect of Coral Exoskeleton

Incisions were made through the skull and into the two cortical hemispheres of adult (3 months old) mice using a needle that contained either particulate coral skeleton (right hemisphere) or 100 micrometer glass beads (left hemisphere) as a control.

Under such conditions, the particulate coral exoskeleton or glass bead is present in the tissue while the wound is created, enabling assessment of the effect of contact with coral exoskeleton on clot formation.

The animals were sacrificed at different times, the brains sectioned near the wound and photographed. FIGS. 2A-2F are reproductions of the photographs.

In FIG. 2E is shown the brain of a mouse sacrificed 3 hours after wounding and concomitant implantation of the particles. Extensive clot formation is observed around the wound contacting the glass beads [no CS] but not around the wound contacting the particulate coral exoskeleton [+CS].

In FIG. 2F, is shown the brain of a mouse sacrificed 3 days after wounding and concomitant implantation of the particles. The wound contacting the glass beads [no CS] shows the presence of a substantial clot while the wound contacting the particulate coral exoskeleton [+CS] shows no blood clot.

In FIGS. 2A-2D are shown the brain of a mouse sacrificed 3 days after wounding and concomitant implantation of the particulate coral exoskeleton, at various magnifications. The wound in contact with the glass particles [no CS] shows the presence of a substantial clot while the wound in contact with the particulate coral exoskeleton [+CS] shows no blood clot.

### Example 4

### In Vivo Decoagulation of Blood by Coral Exoskeleton

Incisions were made through the skull and into the two cortical hemispheres of an adult (3 months old) mouse using a needle. About 20 minutes later, after bleeding from the wound was observed, particles were inserted into the wounds: particulate coral skeleton (left hemisphere) or 100 micrometer glass beads (right hemisphere) as a control.

Under such conditions, the particulate coral exoskeleton or glass bead is contacted with the wound after formation of clots, allowing assessment of the clot-dissolving effect of contact with coral exoskeleton.

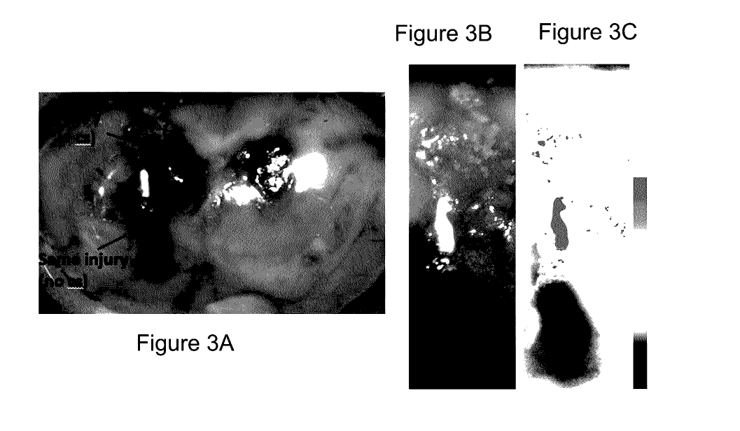

The animal was sacrificed after 3 days, the brain sectioned near the wound and photographed. FIGS. 3A-3C are reproductions of the photographs.

In FIG. 3A is seen the presence of a substantial blood clot in the wound in contact with the glass bead.

In FIGS. 3A (both wounds) and 3B (magnification of the wound with particulate coral exoskeleton) is seen that in the vicinity of particulate coral exoskeleton there is no blood clot, but, at a greater distance from the coral exoskeleton, in the same wound, there is a substantial blood clot.

To more clearly observe the differences, the middle frame (a color photograph, herein reproduced in monochrome) was converted to a monochrome image depicted in FIG. 3C.

Specifically, each pixel in FIG. 3C was assigned an intensity dependent on the color of the corresponding pixel in FIG. 3B, where the color corresponding to a blood clot was assigned an intense (dark) pixel value and the color corresponding to brain tissue without blood clot was assigned a pale (light colored) pixel value. Accordingly, FIG. 3C clearly demonstrates how contact of coral exoskeleton with a nervous tissue wound that already included blood clots leads to substantial elimination of already-formed blood clots in proximity to the coral exoskeleton.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

Citation or identification of any reference in this application shall not be construed as an admission that such reference is available as prior art to the invention.

1. A method for influencing coagulation of blood comprising contacting an effective amount of calcium carbonate with blood, thereby influencing coagulation of the blood.

2. The method of claim 1, wherein said calcium carbonate comprises crystalline calcium carbonate.

3. The method of claim 1, wherein said calcium carbonate comprises aragonite.

4. The method of claim 1, wherein said calcium carbonate comprises acellular coral exoskeleton.

5. The method of claim 4, wherein said coral exoskeleton is provided in dimensions which maintain a porous coral exoskeleton structure.

6-7. (canceled)

8. The method of claim 1, wherein at least some of said blood is unclotted, and said contacting said calcium carbonate inhibits coagulation of said unclotted blood.

9. The method of claim 1, wherein at least some of said blood is clotted blood, and said contacting said calcium carbonate at least partially dissolves said clotted blood.

10. The method of claim 1, wherein said contacting of said calcium carbonate with said blood is effected in vivo.

11. The method of claim 8, wherein said contacting is effected in an ischemic tissue or a tissue region which is at risk of ischemia.

12. The method of claim 10, wherein said contacting of said calcium carbonate with said blood is effected by applying calcium carbonate to a wound.

13-14. (canceled)

15. The method of claim 1, wherein said calcium carbonate is comprised in a device.

16. The method of claim 15, wherein said device comprising said calcium carbonate is selected from the group consisting of a catheter, a dialysis catheter, a balloon catheter, a stent, a heart valve, a blood vessel bypass, a blood vessel graft, a filter, an artificial heart, a shunt and an embolic protection device.

17-18. (canceled)

19. The method of claim 1, wherein said contacting of said calcium carbonate with said blood is effected ex vivo.

20. The method of claim 19, comprising ex vivo contacting said blood with a device comprising said calcium carbonate.

21. The method of claim 20, wherein said device is selected from the group consisting of an artificial heart, a renal dialysis device, a pheresis device and transfusion filter.

22. The method of claim 1, wherein said contacting of said calcium carbonate with said blood is effected in vitro.

23. The method of claim 22, comprising in vitro contacting said blood with a device comprising said calcium carbonate.

24. The method of claim 23, wherein said device is selected from the group consisting of blood collection or storage device, a conduit, a flask, a bottle, a dish, a petri dish, a plate, a multiwell plate, a test tube and a blood transfusion bag.

25. A device for contacting blood, comprising isolated, disinfected aragonite.

26. The device of claim 25, wherein said aragonite comprises coral exoskeleton.

27. The device of claim 25, configured for placement inside the body of a living animal and contact with blood in vivo or configured for contact with blood ex-vivo.

28. The device of claim 25, selected from the group consisting of a catheter, a dialysis catheter, a balloon catheter, an embolic protection device, a stent, a heart valve, a blood vessel bypass, a blood vessel graft, a filter, an artificial heart, a shunt, an embolic protection device, an artificial heart, renal dialysis device and transfusion filter, a blood collection or storage device, a conduit, a flask, a bottle, a dish, a petri dish, a plate, a multiwell plate, a test tube and a blood transfusion bag.

29-30. (canceled)

31. A pharmaceutical composition for inhibiting the coagulation of blood and/or dissolving clotted blood, comprising particulate calcium carbonate and a pharmaceutically acceptable carrier.

32. The pharmaceutical composition of claim 31, wherein said particulate calcium carbonate comprises aragonite.

33. The pharmaceutical composition of claim 31, wherein said particulate calcium carbonate comprises coral exoskeleton.

34. (canceled)

35. The pharmaceutical composition of claim 31, further comprising an additional active ingredient.

36. A kit for inhibiting the coagulation of blood, comprising an effective amount of medical grade, disinfected aragonite.

37. (canceled)

38. The method of claim 1, wherein said calcium carbonate comprises coral exoskeleton from a coral of the Porites species.

39. The method of claim 38, wherein said coral is Porites lutea.

40. The method of claim 1, wherein said calcium carbonate, aragonite or coral exoskeleton is particulate calcium carbonate, aragonite or coral exoskeleton.

41. The method of claim 40, wherein said particulate calcium carbonate or aragonite or coral skeleton has an average particle diameter of between about 1 micrometer and about 3 mm.

42. The method of claim 40, wherein said particulate calcium carbonate, aragonite or coral exoskeleton comprises particles having an average particle diameter in the range of 0.1-10, 0.25-8.0, 0.5-5.0, 0.8-2.5, 1.0-3.0, or 0.05-0.5, 0.5-0.75, 0.75-1.0, 1.0-2.0, 2.0-5.0, 3.0-10.0 millimeters.

43. The method of claim 40, wherein said particulate calcium carbonate, aragonite or coral exoskeleton comprises particles having an average particle diameter of at least 0.5, at least 0.75, at least 1.0, at least 1.25, at least 1.5 or at least 2.0 millimeters.

44. The method of claim 40, wherein said particulate calcium carbonate, aragonite or coral exoskeleton comprises particles having an average particle diameter in the range of 0.5 micrometers to 10 millimeter, 1.0 micrometer to 100 micrometers, 2.0 micrometers to 75 micrometers, 5.0 micrometer to 50 micrometers, 7.5 micrometer to 30 micrometers, 5.0 micrometer to 20 micrometers, 50 micrometers to 150 micrometers and 1.0 micrometer to 10.0 micrometers.

45. The method of claim 40, wherein said particulate calcium carbonate, aragonite or coral exoskeleton comprises particles having an average particle diameter of at least 0.5, at least 0.75, at least 1.0, at least 2, at least 3, at least 5, at least 7.0, at least 10.0, at least 12.5, at least 15, at least 20, at least 35, at least 50, at least 75, at least 100 or at least 250 micrometers.
